(19) United States
(12) Patent Application Publication
(10) Pub. No.: US 2019/0285488 A1
(43) Pub. Date: Sep. 19, 2019

(54) CALIBRATING A HEAT FLUX SENSOR FOR MEASURING BODY TEMPERATURE OF AN INDIVIDUAL

(71) Applicant: JonDeTech Sensors AB, Stockholm (SE)

(72) Inventors: Patrik Lundström, Stockholm (SE); Michael Olsson, Stockholm (SE); Mikael Lindeberg, Johanneshov (SE)

(21) Appl. No.: 16/301,946

(22) PCT Filed: May 23, 2017
(86) PCT No.: PCT/SE2017/050549

§ 371 Date: Nov. 15, 2018

(30) Foreign Application Priority Data
May 27, 2016 (SE) 1650733-7

(51) Int. Cl. G01K 15/00; G01K 13/00; A61B 5/01

(52) U.S. Cl. CPC G01K 15/005; G01K 13/002; A61B 2562/0271; A61B 2560/0223; A61B 2560/0252; A61B 5/01

(57) ABSTRACT

A method of calibrating a heat flux sensor for measuring body temperature of an individual, and a heat flux sensor. The method includes measuring heat flux with the heat flux sensor applied to a part of the body of the individual, acquiring a reference temperature value for the heat flux sensor, the reference temperature being measured at a side of the heat flux sensor facing away from the body, and acquiring a body temperature value for the individual. Further, the method comprises determining an overall heat transfer coefficient for the sensor and the individual by using the measured heat flux, the acquired reference temperature value, and the acquired body temperature value.

### TECHNICAL FIELD

The invention relates to a method of calibrating a heat flux sensor for measuring body temperature of an individual, and a heat flux sensor.

### BACKGROUND

In the art, measuring body temperature of mammals, and in particular human beings, has been a long-standing problem.

Invasive methods are well-known, such as rectal, oral or tympanic measurement, but have a tendency of causing discomfort to the individual begin subjected to the invasive temperature measurement. Further, it must be ensured that a measuring probe is properly positioned upon performing invasive temperature measurement. Moreover, body temperature varies slightly depending on the part of the body being subjected to the measurement.

Therefore, non-invasive body temperature measurement methods are preferred. Non-invasive methods of measuring body temperature are even further brought to the fore with the advent of various types of wearables, such as smartwatches, fitness trackers, health monitoring devices, digital plasters, garments, etc.

### SUMMARY

An object of the present invention is to solve theses problems in the art and to provide a method of non-invasive measurement of body temperature.

This object is attained in a first aspect of the invention by a method of calibrating a heat flux sensor for measuring body temperature of an individual. The method comprises measuring heat flux with the heat flux sensor applied to a part of the body of the individual, acquiring a reference temperature value for the heat flux sensor, the reference temperature being measured at a side of the heat flux sensor facing away from the body, and acquiring a body temperature value for the individual. Further, the method comprises determining an overall heat transfer coefficient for the sensor and the individual by using the measured heat flux, the acquired reference temperature value, and the acquired body temperature value.

This object is attained in a second aspect of the invention by a heat flux sensor configured to measure body temperature of an individual. The heat flux sensor is arranged to measure heat flux with the heat flux sensor applied to a part of the body of the individual, acquire a reference temperature value for the heat flux sensor, the reference temperature being measured at a side of the heat flux sensor facing away from the body, and acquire a body temperature value for the individual. The heat flux sensor is further arranged to determine an overall heat transfer coefficient for the sensor and the individual by using the measured heat flux, the acquired reference temperature value, and the acquired body temperature value.

Advantageously, by measuring a voltage output of the sensor, the heat flux can be determined. Thereafter, a reference temperature value is measured at an upper side of the heat flux sensor, for instance using a thermistor.

The overall heat transfer coefficient for the sensor and the individual on which it is arranged is calculated based on the heat flux and the difference between the reference temperature and the body temperature Tc.

Advantageously, with the invention, the overall heat transfer coefficient h is calibrated by either:
(a) assuming the body temperature to be Tc=37° C. (or whatever value is considered to best represent a “normal” body temperature), or
(b) measuring the body temperature of the individual.

Using either option (a) or (b), the overall heat transfer coefficient is determined, and the heat flux sensor has advantageously been calibrated. This heat transfer coefficient can be stored for subsequent use.

Option (b) may advantageously be preferred if the sensor for instance is implemented in a wearable such as a smartwatch or a health bracelet, or a smart phone or tablet being personal to, and thus only to be used by, the individual.

In another scenario, where the sensor is to be used by a larger group of individuals, perhaps only once or twice for each individual in the group, it may advantageously be preferred to set Tc=37° C. as proposed in option (a).

In an embodiment, body temperature of the individual is advantageously measured using the determined overall heat transfer coefficient, a measured heat flux and an acquired reference temperature value. The sensor device may be re-calibrated if required, thus acquiring an updated overall heat transfer coefficient.

In an embodiment, the sensor is advantageously implemented in a smart phone or a wearable, which comprises an app operable by a user to cause the smart phone/wearable to perform the calibration process described hereinabove, and further to measure a body temperature of the individual.

For instance, it may be envisaged that the user presses “calibrate” on the temperature app of the smart phone, wherein a processing unit of the phone reads the voltage output from the heat flux sensor and determines heat flux accordingly. Thereafter, the processing unit reads the sensor reference temperature from the thermistor of the sensor. The processing unit further acquires the body temperature, for instance from a server, or by acquiring a pre-stored value from its memory, or by the user entering a temperature value via the app.

Finally, the processing unit advantageously determines the heat transfer coefficient based on the measured heat flux, the acquired reference temperature value, and the acquired body temperature value and stores the value in the memory.

Subsequently, after having calibrated the sensor for the combination of the sensor and individual properties of the user as regards for instance skin thickness, fat, body tissue, the user presses “measure temp” of the app, wherein the processing unit measures the sensor voltage output and the reference temperature using the thermistor, and advantageously utilizes the stored heat transfer coefficient to measure the body temperature of the user.

In yet an embodiment, the sensor (or the mobile phone/wearable) is advantageously capable of communicating with a remotely located device, such as a server, for reporting measured body temperatures.

Further provided is a computer program comprising computer-executable instructions for causing the heat flux sensor to perform the method according to the first aspect of the invention, when the computer-executable instructions are executed on a processing unit included in, or in connection to, the heat flux sensor.

Further provided is a computer program product comprising a computer readable medium, the computer readable medium having the computer program of the processing unit embodied thereon.

Further embodiments will be described in the detailed description.

Generally, all terms used in the claims are to be interpreted according to their ordinary meaning in the technical field, unless explicitly defined otherwise herein. All references to “a/an/the element, apparatus, component, means, step, etc.” are to be interpreted openly as referring to at least one instance of the element, apparatus, component, means, step, etc., unless explicitly stated otherwise. The steps of any method disclosed herein do not have to be performed in the exact order disclosed, unless explicitly stated.

### BRIEF DESCRIPTION OF THE DRAWINGS

The invention is now described, by way of example, with reference to the accompanying drawings, in which:

FIG. 1 illustrates a body temperature measurement by applying a heat flux sensor according to an embodiment of the invention to a part of the body of an individual being subjected to the temperature measurement;

FIG. 2 shows a flowchart illustrating a method of calibrating a heat flux sensor for measuring body temperature of an individual according to an embodiment of the invention;

FIG. 3 shows a heat flux sensor being equipped with a microprocessor and a communication interface 12 according to an embodiment of the invention;

FIG. 4 illustrates an embodiment where the heat flux sensor is implemented in wearable;

FIG. 5 illustrates a further embodiment, where the sensor is implemented within a smart phone;

FIG. 6 illustrates yet a further embodiment, where the sensor is implemented within a smart phone; and

FIG. 7 illustrates a heat flux sensor according to an embodiment.

### DETAILED DESCRIPTION

The invention will now be described more fully hereinafter with reference to the accompanying drawings, in which certain embodiments of the invention are shown. This invention may, however, be embodied in many different forms and should not be construed as limited to the embodiments set forth herein; rather, these embodiments are provided by way of example so that this disclosure will be thorough and complete, and will fully convey the scope of the invention to those skilled in the art. Like numbers refer to like elements throughout the description.

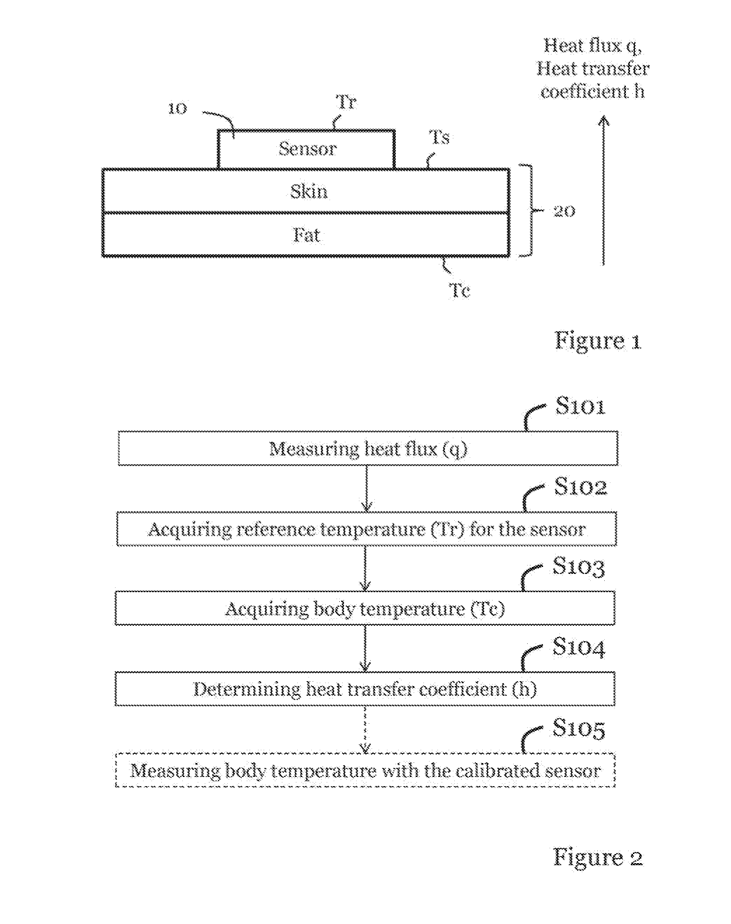

FIG. 1 illustrates a body temperature measurement by applying a heat flux sensor 10 according to an embodiment of the invention to a part of the body of an individual 20 being subjected to the temperature measurement.

As is illustrated in FIG. 1, skin temperature measured at a bottom side of the sensor 10 is denoted Ts, while ambient temperature measured at an upper side of the sensor 10 is denoted Ta. This is for illustration only; it is noted that it is not necessary to measure skin temperature Ts in the embodiment discussed in the below.

Body temperature is the temperature of the individual 20 underneath skin and fat tissue and is denoted Tc.

Reference is further made to FIG. 2 showing a flowchart illustrating a method of calibrating a heat flux sensor for measuring body temperature of an individual according to an embodiment.

Now, in step S101, heat flux is measured with the heat flux sensor 10 applied to a part of the body of the individual 20.

Heat flow or flux, q, is measured as:

$$q = \frac{{V}_{sen}}{{E}_{sen}} \qquad ( 1 )$$

where Vsen is the sensor voltage output and Esen is a known calibration constant, specific for the individual sensor 10. Hence, heat flux q is calculated with equation (1).

The heat flux q is thus indirectly measured using the sensor voltage output Vsen and the known calibration constant Esen of the sensor.

In step S102, temperature Tr at an upper side of the sensor 10, i.e. the side of the sensor 10 facing away from the body of the individual 20, is measured for reference. This may be undertaken by using a temperature sensor, such as e.g. a thermistor, arranged at the heat flux sensor 10.

Now, the so called heat transfer coefficient h is indeed known for the sensor 10, but unknown for the sensor 10 and the individual 20 combined due to individual variations among human beings (or animals).

The overall heat transfer coefficient h is calculated as:

$$h = \frac{q}{Δ T} = \frac{q}{Tc - Tr} \qquad ( 2 )$$

As can be concluded, the overall heat transfer coefficient for the sensor 10 and the individual 20 in combination depends on the heat flux q and the difference between the reference temperature Tr (i.e. the temperature at the upper side of the sensor 10) and the body temperature Tc.

Advantageously, with the invention, the overall heat transfer coefficient h is calibrated by either:
(c) assuming the body temperature to be Tc=37° C. (or whatever value is considered to best represent a “normal” body temperature), or
(d) measuring the body temperature of the individual.

It is known that different individuals have different body temperatures, and the “normal” body temperature, referred to as normothermia, varies in the range of 36.5-37.5° C.

Hence, measuring body temperature Tc of the individual 20 on which the sensor 10 is to be applied once and for all will give a more accurate result, assuming that the individual has a normothermia of, say, 36.5° C.

Option (b) may advantageously be preferred if the sensor 10 for instance is implemented in a wearable such as a smartwatch or a health bracelet, a smart phone or tablet being personal to, and thus only to be used by, the individual 20.

In another scenario, where the sensor is to be used by a larger group of individuals, perhaps only once or twice for each individual in the group, it may advantageously be preferred to set Tc=37° C. as proposed in option (a).

With the measured or estimated body temperature Tc acquired in step S103, equation (2) may advantageously be used for calibrating the sensor 10 for use with this particular individual 20 by determining the overall heat transfer coefficient h, as is finally done in step S104.

This process of calibrating the sensor 10 for use with the individual 20 may advantageously be frequently repeated, for instance due to increase or decrease of fat tissue of the individual.

In an embodiment, following the calibration of the sensor 10 in step S101-S104, the body temperature Tc of the individual 20 may continuously be measured in step S105 taking into account sensor measurements of the heat flux using equation (2) in modified form:

$$Tc = \frac{q}{h} + Tr \qquad ( 3 )$$

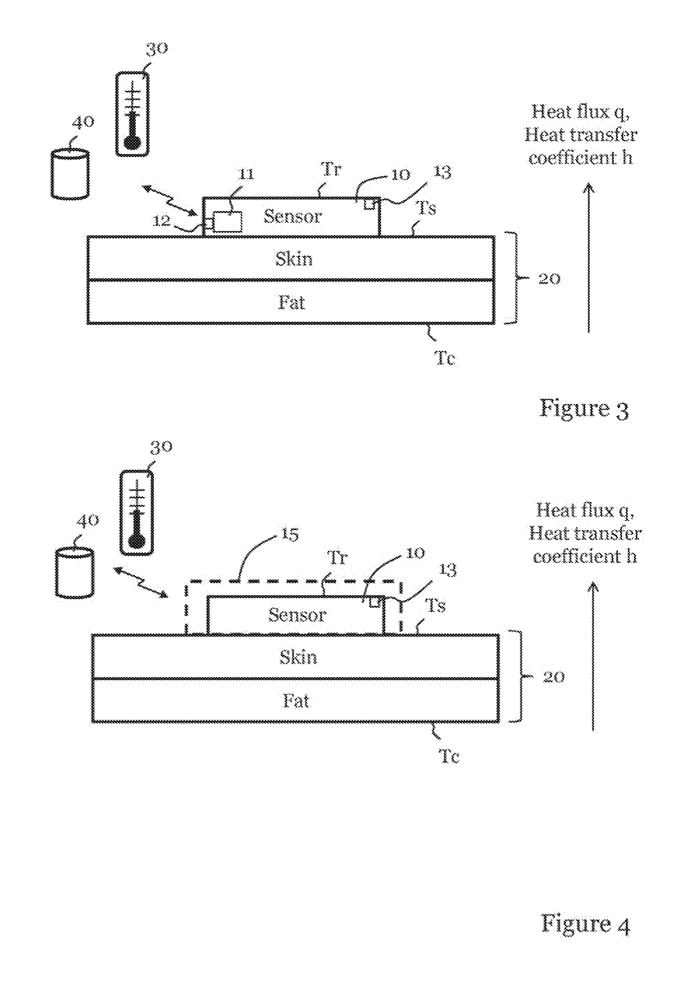

FIG. 3 shows a sensor 10 being equipped with a processing device 11, such as a microprocessor, for performing calculations according to equations (1)-(3), and even with a communication interface 12, wired or wireless, for transmitting/receiving data to/from a remote location according to an embodiment of the invention. The sensor 10 of FIG. 3 is further equipped with a thermistor 13 for measuring the reference temperature Tr.

The microprocessor 11 may be integrated with the sensor 10, or arranged on a printed circuit board shared with the sensor 10.

In such an embodiment, it can be envisaged, in particular in the light of an ever emerging Internet of Things (IoT) with various connected sensors and devices, that the microprocessor 11 of the sensor 10 receives, from an IoT enabled thermometer 30 remotely located from the sensor 10, the body temperature Tc of the individual via the wireless interface 12, which previously has been measured by the thermometer 30.

Subsequently, the microprocessor 11 calibrates the sensor 10 as described in steps S101-S104 to attain the heat transfer coefficient h or, if the calibration already has been performed, measures the body temperature Tc of the individual 20 by utilizing equation (3).

As is further illustrated in FIG. 3, it may be envisaged that the sensor device to in an embodiment submits any measurement results to a remotely located device, such as a server 40, for further analysis and/or processing.

Further, it may be envisaged that the measured body temperature Tc for each of a population of individuals is centrally held in a database stored at the remote server 40, wherein the microprocessor 11 fetches the measured body temperature Tc for this particular individual 20 via the wireless interface 11 from the database at the server 40 when required. As an alternative, it is envisaged that the individual herself can enter the measured body temperature Tc via the interface 11. In such a scenario, it is particularly advantageous if the interface 11 is a graphical user interface, for instance a touch screen.

FIG. 4 illustrates an embodiment where the sensor 10 is implemented in wearable 15, such as a smartwatch, a health bracelet, a fitness tracker, etc. The sensor 10 may even be implemented with a garment, such as a shirt, in a digital plaster or a patch similar to wound patches.

The ambient temperature Tr of the sensor device to is the temperature at the upper side of the sensor device, i.e. a temperature internal to the wearable 15, measured for instance by the thermistor 13. Further, the wearable 15 already comprises intelligence in the form of a microprocessor, memory, a communication interface, etc.

Again, the heat flux is measured by the sensor 10 according to equation (1), and the wearable 15 calibrates the overall heat transfer coefficient using equation (2).

The body temperature Tc is either estimated or measured as previously discussed, and after having been calibrated, the sensor 10 can measure body temperature using equation (3).

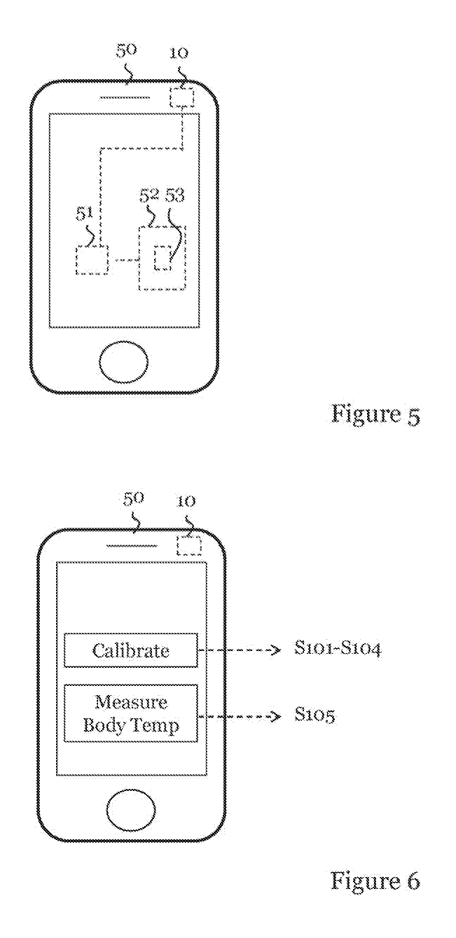

FIG. 5 illustrates a further embodiment, where the sensor 10 is implemented within a smart phone 50. Hence, in accordance with the method of measuring temperature as described above, a user may place the back side of the smart phone 50 against a part of her body and start an app on the smart phone 50 for measuring her body temperature, wherein the body temperature is measured and presented on the screen of the smart phone 50.

With further reference to FIG. 5, some steps of the method according to embodiments are in practice performed by a processing unit 51 embodied in the form of one or more microprocessors arranged to execute a computer program 53 downloaded to a suitable storage medium 52 associated with the microprocessor 51, such as a Random Access Memory (RAM), a Flash memory, a hard disk drive, a cloud service or other information storage devices. The processing unit 51 is arranged to cause the sensor 10 to carry out measurements according to embodiments when the appropriate computer program 53 comprising computer-executable instructions is downloaded to the storage medium 52 and executed by the processing unit 51. The storage medium 52 may also be a computer program product comprising the computer program 53. Alternatively, the computer program 53 may be transferred to the storage medium by means of a suitable computer program product, such as a Digital Versatile Disc (DVD) or a memory stick. As a further alternative, the computer program 53 may be downloaded to the storage medium 52 over a network. The processing unit 51 may alternatively be embodied in the form of a digital signal processor (DSP), an application specific integrated circuit (ASIC), a field-programmable gate array (FPGA), a complex programmable logic device (CPLD), etc.

With reference to FIG. 6 and further to the flowchart of FIG. 2, the user presses “calibrate” on the temperature app of the smart phone 50, wherein the processing unit 51 reads the voltage output Vsen from the heat flux sensor 10 and determines heat flux in step S101 using equation (1). Thereafter, the processing unit 51 reads the sensor reference temperature from the thermistor 13 according to step S102. The processing unit 51 further acquires the body temperature, for instance from the server 40, in step S103, or by acquiring a pre-stored value from the memory 52, or by the user entering a temperature value via the app.

Finally, the processing unit 51 determines the heat transfer coefficient in step S104 using equation (2), and stores the value in the memory 52. This process may be repeated on a continuous basis, such as once a week, either by the user operating the “calibrate” icon of the app, or the mobile phone 50 automatically performing a temperature re-calibration procedure.

Subsequently, after having calibrated the sensor 10 for the combination of the sensor and individual properties of the user as regards for instance skin thickness, fat, body tissue, the user may operate the “Measure temp” icon of the app, wherein the processing unit 51 measures the sensor voltage output Vsen and the reference temperature Tr using the thermistor 13, and finally utilizes equation (3) with the stored heat transfer coefficient h to measure the body temperature Tc of the user as described in step S105.

In an embodiment, the smart phone 50 (or the previously described wearable 15) wirelessly submits measured body temperature values to the central server 40 to keep a record, the server 40 being located for instance at a medical institute). In yet an embodiment, measured body temperature values are stored locally with the app such that the user may keep a record and follow trends by consulting the app for measured body temperature values.

In case a wearable, such as a digital plaster, comprises the sensor 10 according to an embodiment of the invention, it may be envisaged that the plaster continuously measures and stores body temperature values of the user, and notifies the user, for example by means of an audio alert, about a trend of the measured values, such as if the measured values indicates that the user is catching fever. This is particularly advantageous in case the digital plaster is applied to a child, where e.g. a digital plaster sounds an alarm if the body temperature of the child exceeds 37° C. thereby notifying a parent of the measured body temperature.

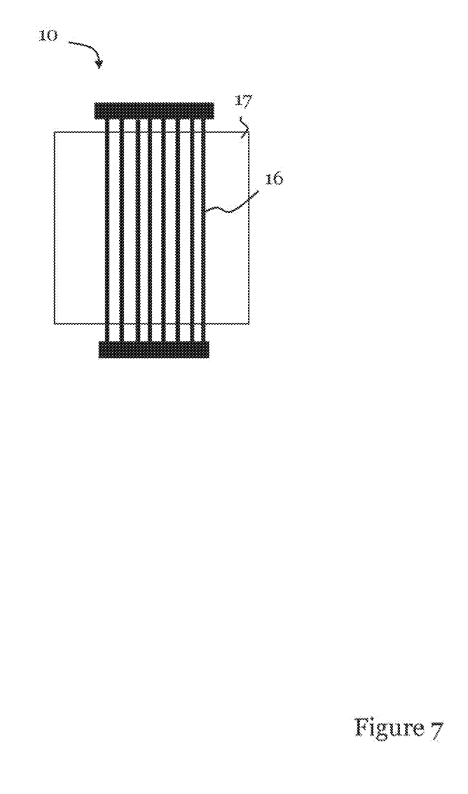

FIG. 7 illustrates a heat flux sensor 10 according to an embodiment. In the sensor, a plurality of nanowires 16 being 500-700 nm in diameter are encapsulated by a plastic substrate 17.

The nanowires 16 generate the voltage Vsen from the temperature difference between an upper and lower side of the sensor 10. This is achieved by using a unique combination of different metals in the nanowires 16. The production process must be highly precise in terms of etching and plating in order to achieve adequate connection between the different metal materials inside each nanowire 16.

The invention has mainly been described above with reference to a few embodiments. However, as is readily appreciated by a person skilled in the art, other embodiments than the ones disclosed above are equally possible within the scope of the invention, as defined by the appended patent claims.

1. A method of calibrating a heat flux sensor for measuring body temperature of an individual, comprising the steps of:
measuring heat flux with the heat flux sensor applied to a part of the body of the individual;
acquiring a reference temperature value for the heat flux sensor, the reference temperature being measured at a side of the heat flux sensor facing away from the body;
acquiring a body temperature value for the individual; and
determining an overall heat transfer coefficient for the sensor and the individual by using the measured heat flux, the acquired reference temperature value, and the acquired body temperature value.

2. The method of claim 1, wherein the acquiring of a reference temperature value for the sensor comprises:
measuring the reference temperature value for the sensor with a temperature sensor.

3. The method of claim 1, wherein the acquiring of a body temperature value comprises:
estimating a body temperature value for the individual for determining the overall heat transfer coefficient.

4. The method of claim 1, wherein the acquiring of a body temperature value comprises:
receiving, from a remote location, a measured body temperature value for the individual for determining the overall heat transfer coefficient.

5. The method of claim 1, further comprising:
measuring a body temperature value of the individual using the determined overall heat transfer coefficient, a measured heat flux and an acquired reference temperature value.

6. A heat flux sensor system configured to measure body temperature of an individual, the heat flux sensor system comprising a heat flux sensor and a processing unit configured to:
measure heat flux with the heat flux sensor applied to a part of the body of the individual;
acquire a reference temperature value for the heat flux sensor, the reference temperature being measured at a side of the heat flux sensor facing away from the body;
acquire a body temperature value for the individual; and
determine an overall heat transfer coefficient for the sensor and the individual by using the measured heat flux, the acquired reference temperature value, and the acquired body temperature value.

7. The heat flux sensor system of claim 6, further comprising a temperature sensor configured to measure the reference temperature value for the sensor.

8. (canceled)

9. The heat flux sensor system of claim 6, wherein the processing unit is further configured to estimate the body temperature value for the individual for determining the overall heat transfer coefficient.

10. The heat flux sensor system of claim 6, wherein the processing unit comprises a communication interface configured to receive a measured body temperature value for the individual for determining the overall heat transfer coefficient.

11. The heat flux sensor system of claim 6, wherein the processing unit is further configured to measure a body temperature value of the individual using the determined overall heat transfer coefficient, a measured heat flux and an acquired reference temperature value.

12. (canceled)

13. The heat flux sensor system of claim 6, wherein the heat flux sensor and the processing unit are integrated into a smart phone.

14. The heat flux sensor system wearable of claim 13, wherein the processing unit is configured to:
notify the individual about measured body temperature values.

15. The heat flux sensor system of claim 13, further comprising an app operable by a user to cause the smart phone to perform the method of:
measuring heat flux with the heat flux sensor applied to a part of the body of the individual;
acquiring a reference temperature value for the heat flux sensor, the reference temperature being measured at a side of the heat flux sensor facing away from the body;
acquiring a body temperature value for the individual; and
determining an overall heat transfer coefficient for the sensor and the individual by using, the measured heat flux, the acquired reference temperature value, and the acquired body temperature value.

16. A computer program comprising computer-executable instructions stored in a non-transient computer-readable medium, the computer-executable instructions being configured to cause a device, when the computer-executable instructions are executed on a processing unit included in the device, to:
measure a heat flux with the heat flux sensor applied to a part of the body of the individual;
acquire a reference temperature value for the heat flux sensor, the reference temperature being measured at a side of the heat flux sensor facing away from the body;
acquire a body temperature value for the individual; and
determining an overall heat transfer coefficient for the sensor and the individual by using the measured heat flux, the acquired reference temperature value, and the acquired body temperature value.

17. (canceled)
